(19) United States
(12) Patent Application Publication
(10) Pub. No.: US 2026/0014372 A1
(43) Pub. Date: Jan. 15, 2026

(54) METHODS AND SYSTEMS FOR TRANSDERMALLY STIMULATING A MUSCLE

(71) Applicant: ZANSORS LLC, Arlington, VA (US)

(72) Inventors: Tim White, Pensacola, FL (US); Maurice Jackson, COLORADO SPRINGS, CO (US); Ancha Baranova, Annandale, VA (US); Baabi Das, Arlington, VA (US)

(73) Assignee: Zansors LLC, Arlington, VA (US)

(21) Appl. No.: 18/994,696

(22) PCT Filed: Sep. 26, 2023
(86) PCT No.: PCT/US2023/033700

§ 371 Date: Jan. 15, 2025

Related U.S. Application Data
(60) Provisional application No. 63/392,916, filed on Jul. 28, 2022.

(51) Int. Cl. A61N 1/36; A61N 1/04

(52) U.S. Cl. CPC A61N 1/36003; A61N 1/0452; A61N 1/0484; A61N 1/0492; A61N 1/36034

(57) ABSTRACT

The present invention discloses methods and systems for transdermally stimulating a muscle. The method comprising or including: a) contacting a surface of the skin in proximity to a target muscle with at least two electrodes; b) energizing the at least two electrodes to expose the target muscle to a first voltage having a first polarity for a first time period; and c) energizing the at least two electrodes to expose the target muscle to a second voltage having a second polarity, opposite the first polarity, for a second time period. The system and method enable to vary the polarity of the voltage to which the target muscle is exposed is sufficient to stimulate the target muscle.

### CROSS-REFERENCE TO RELATED APPLICATIONS

The present application claims priority to PCT International application No. PCT/US2023/033700 filed on 26 Sep. 2023, which claims the benefit of, and priority to, U.S. Provisional Patent Application with Ser. No. 63/392,916, filed on Jul. 28, 2022, the disclosure of which are incorporated herein by reference in its entirety.

### BACKGROUND OF THE INVENTION

### A. Technical Field

The present invention relates to the electrical stimulation of muscles to, for example, treat or counter-act the effects of paralysis. Specifically, aspects of the present invention vary the polarity of the applied electrical stimulation to overcome the deficiencies and disadvantages of the existing art.

### B. Description of related art

Electrical stimulation of the body for a variety of intentions has been attempted since at least the 1700s. In the 1780s, Galvani first discovered that the muscles of dead frogs twitched when exposed to an electric spark. Benjamin Franklin is reported to have used electric shock in an attempt to treat pain and other ailments. In the 1890s, Nicola Tesla experimented with treating the human body with electricity to address a variety of ailments. In the 1970s, Transcutaneous Electrical Nerve Stimulation (TENS) was developed as a means for stimulating human nerves with electrical current.

In the 1960s, Functional Electrical Stimulation (FES) was introduced as a means for stimulating human muscles with electricity to generate bodily movements, for example, in patients suffering from paralysis. Since that time, FES has been developed and used in a broad range of medical applications, including for treating the effects of spinal cord injury, stroke, and muscle sclerosis, among other ailments.

However, it is recognized in the art that conventional FES treatment is limited by the pain that can be induced in a patient when FES is applied to a patient. For example, the application of relatively high voltages, for instances, voltages greater than 50 volts can be painful to the patient. Aspects of the present invention address this undesirable disadvantage of the prior art methods.

It is generally understood in the art that, when exposed to an electric voltage, skin can be viewed electrically as an electric circuit having a capacitor and a resistor in series. For example, Kim, et al. (2010) [“A New Method for Non-Invasive Measurement of Skin in the Low Frequency Range”, Kim, et al., PubMed, September 2010, the disclosure of which is included herein], describe an investigation of the electrical characteristics of skin.

Based upon this present understanding, it is believed that the capacitor-like electrical character of the skin behaves like any capacitor, that is, when exposed to a DC voltage, current flows and the capacitor is charged, and then the current dissipates to zero as one side of the capacitor reaches its positive charge. According to this understanding, the “charging” of the “capacitor” of the skin can result in local polarization of the skin about the area of contact of an electrode with the skin. Furthermore, it is believed that this local polarization of the skin can undesirably result in the flow of ions (for example, positive ions) from the electrode into the skin and/or from the skin to the electrode. Though it is presently unclear what the long-term effects of such ion flows may be, it is generally believed to be possibly deleterious, and possibly harmful, to the patient being treated. According to one aspect of the invention, this polarization is minimized or prevented where ion flow between electrode and skin is minimized or prevented.

In a further analogy of the capacitor-like character of skin, it is speculated that, when the voltage applied to the skin by an electrode varies, the frequency at which a voltage is varied affects the impedance of the skin. It is believed that the impedance of the skin to the voltage can be manifest as pain by the patient being treated. Thus, it is speculated that reducing the impedance of the skin can reduce any pain that is likely to be felt by the patient. For example, if a given current is required to generate a desired level of muscle stimulation, then lowering the impedance of the current path through the skin by varying the voltage can reduce the voltage needed to provide the required current. Thus, it is believed, varying the voltage and lowering the voltage can possibly reduce the potential pain because the voltage is lower, while providing the desired muscle stimulation.

For example, it is known in the art that the capacitive impedance, XC, of a capacitor, such as the skin, is a function of voltage frequency, f, and can be expressed as shown in Equation 1.

$$XC = 1 / ( 2 π f C ) \qquad Equation 1$$

C is the capacitance of capacitor, that is, the skin, in farads. Examination of Equation 1 indicates that impedance XC of the skin increases, and thus pain may increase, as the frequency f of the applied voltage decreases. That is, the lower the frequency f, the higher the pain for the patient. In addition to other advantages of the present invention, aspects of the invention provide for the application of variation in voltage at relatively higher frequencies, for example, 1 kHz or higher, that can minimize or prevent the likelihood of exposing the patient being treated to pain.

In addition, according to aspects of the invention, and also with regard to Equation 1, where aspects of the invention can provide muscle stimulation voltages at higher frequencies, in addition to reducing the likelihood of pain, aspects of the invention can reduce the impedance of the skin such that the electric current created by the applied voltage can more effectively access and penetrate the muscle being targeted for stimulation.

These and other advantages and benefits of the invention, in its many aspects, will be apparent upon review of the summary of the invention below.

### SUMMARY OF THE INVENTION

As described herein, the present invention comprises methods and systems for providing alternating DC voltages to the skin adjacent a muscle being stimulated in which the polarity of the DC voltages is varied. It is believed that the changing of the polarity of the applied voltage minimizes or prevents the polarizing effects, reduces the likelihood of inducing pain, and increases the effective stimulation of the muscle being stimulated. Moreover, aspects of the invention can provide comparable performance to prior art muscle stimulation methods while reducing the energy required to achieve the prior art performance.

One embodiment of the invention is a method for stimulating a muscle, the method comprising or including: a) contacting a surface of the skin in proximity to a target muscle with at least two electrodes; b) energizing the at least two electrodes to expose the target muscle to a first voltage having a first polarity for a first time period; and c) energizing the at least two electrodes to expose the target muscle to a second voltage having a second polarity, opposite the first polarity, for a second time period; wherein varying the polarity of the voltage to which the target muscle is exposed is sufficient to stimulate the target muscle.

In one aspect, the first and second voltages may range from 10 volts-DC [VDC] to 300 VDC. In another aspect, the first voltage and/or the second voltage may be applied at a frequency of between 1 kHz to 10 KHz.

In another aspect, the method may further include, between b) and c), de-energizing the at least two electrodes to expose the target muscle to little or no voltage.

In another aspect, the method may further include, between b) and c), energizing the at least two electrodes to expose the target muscle to a third voltage, different from the first voltage and the second voltage.

In another aspect, the method may further include repeating b) a plurality of times prior to c), and/or repeating c) a plurality of times.

In another aspect, contacting the surface of the skin in proximity to a target muscle may be practiced by mounting the at least two electrodes in one or more packages, for example, in patches, and mounting the one or more packages to the surface of the skin.

Another embodiment of the invention is a system for stimulating a muscle, the system comprising or including: at least two electrodes adapted to contact a surface of the skin in proximity to a target muscle; and an output device having at least two outputs operatively connected to the at least two electrodes, wherein the output device is configured to provide at one of the at least two outlets a first voltage having a first polarity for a first time period, and to provide at another of the at least two outlets a second voltage having a second polarity, opposite the first polarity, for a second time period; wherein varying the polarity of the voltage to which the target muscle is exposed is sufficient to stimulate the target muscle.

In one aspect, the system may further include a high voltage generator operatively connected to the output device to provide the first voltage and the second voltage. For example, the high voltage generator may provide a voltage ranging from 10 VDC to 300 VDC.

In another aspect, the output device may be adapted to vary a frequency of at least one of the first voltage and the second voltage, for example, a frequency ranging from 1 kHz to 10 KHz.

A further embodiment of the invention is a wearable device having the system disclosed herein. In one aspect, the wearable device may comprise a patch having an adhesive adapted to at least temporarily adhere the patch to human skin. In another aspect, the wearable device may be mountable in a garment, gear, or a textile.

### BRIEF DESCRIPTION OF THE DRAWINGS

The subject matter, which is regarded as the invention, is particularly pointed out and distinctly claimed in the claims at the conclusion of the specification. The foregoing and other objects, features, and advantages of the invention will be readily understood from the following detailed description of aspects of the invention taken in conjunction with the accompanying drawings in which:

FIG. 1 is a schematic block diagram of a system that can be used to implement aspects of the invention.

FIG. 2 is a schematic representation of a cross-section of a portion of a body having a muscle that can be stimulated according to aspects of the invention.

FIG. 3 is a detail of the cross section shown in FIG. 2 as identified by Detail 3 in FIG. 2.

FIGS. 4 through 7 are representative illustrations of the variation in voltage polarity that can be provided according to aspects of the invention.

FIG. 8 is a representative illustration of the wearable device with electrodes, controller and power button, and alkaline batteries according to aspects of the invention.

FIG. 9 is a representative illustration of the controller with On/Off buttons and alkaline batteries according to aspects of the invention.

FIG. 10 is a representative illustration of the electrodes according to aspects of the invention.

FIG. 11 exemplarily illustrates an arrangement of components of a wearable device, according to an embodiment of the present invention.

FIG. 12 exemplarily illustrates a segment of interior side of the apparel sewn with the electrodes, according to an embodiment of the present invention.

FIG. 13 exemplarily illustrates a segment of exterior side of the apparel and first conductive threads extending at the exterior side of the apparel, according to an embodiment of the present invention.

FIG. 14 exemplarily illustrates an insulation assembly provided over the first conductive thread, according to an embodiment of the present invention.

FIG. 15 exemplarily illustrates an end portion of the first conductive thread being formed as a loop, according to an embodiment of the present invention

FIG. 16 exemplarily illustrates the first conductive thread passing beyond the insulation assembly for soldering, according to an embodiment of the present invention

### DETAILED DESCRIPTION OF EMBODIMENTS

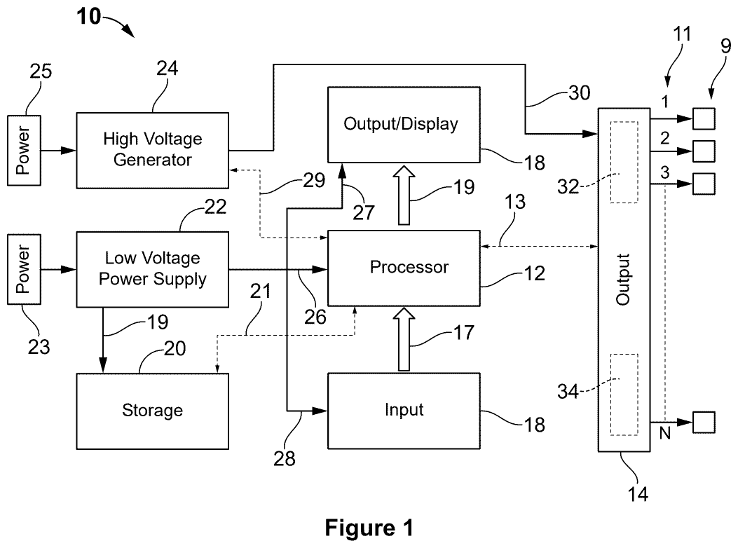

FIG. 1 is a schematic block diagram of a system 10 that can be used to implement aspects of the invention. According to one aspect of the invention, system 10 is adapted to generate and/or moderate and apply a varying voltage to muscle or muscle group to be stimulated. In one aspect, the muscle or muscles to be stimulated may be referred to as “a target muscle” and, according to aspects of the invention, the target muscle is stimulated with an electronic voltage (and/or a corresponding electric current) in order to, for example, encourage contraction of the target muscle. According to aspects of the invention, the contraction of the target muscle by the controlled application of voltage to at least a portion of the target muscle can be used to, among other things, stimulate the target muscle to contract where the patient being treated has lost the ability to stimulate the muscle due to spinal cord injury, stroke, and/or muscle sclerosis, among other ailments, and/or to increase blood flow due to muscle stimulation.

As shown in FIG. 1, system 10 may typically include a processor 12 and an output device 14 adapted to receive instructions via connection 13 from processor 12 and generate a plurality of outputs 11, typically, voltage outputs, to a plurality of electrodes 9 mounted to a body (not shown), for example, a human or non-human body, having a muscle or muscle group to be stimulated. Processor 12 may be any conventional processor, for example, a microprocessor or computer, as known in the art, that contains software, and is adapted to execute the software and generate a control signal 13 forwarded to output device 14. As is typical in the art, processor 12 may be operatively connected to an input device or user interface 16, such as, a keyboard, pointing device, controller, and/or touch screen, and the like, via connection 17, and an output device or display 18 adapted to provide, for example, user feedback or display data generated by processor 12 forwarded via connection 19. System 10 may typically also include a data storage device 20 operatively connected to processor 12 via connection 21, for storing data accessed by processor 12 and/or storing data generated by processor 12. Storage device 20 may comprise a local or remote storage device (for example, a storage location accessed via a network, for instance, available in “the cloud” as known in the art) and may include a fixed or a removable storage medium. For example, in one aspect, storage device 20 may be one or more removable “SD cards” or similar media as known in the art.

As also shown in FIG. 1, system 10 may typically include a low voltage generator 22 powered by a power supply 23 and a high voltage generator 24 powered by a power supply 25. Low voltage power supply 22, for example, a power supply adapted to provide a DC voltage of 0.5 to 5 volts, may power processor 12 via connection 26, may power output or display 18 via connection 27, may power input device 16 via connection 28, and may power storage device 20 via connection 19.

According to aspects of the invention, processor 12 may regulate and control the voltage output by high voltage generator 24, for example, 10 volts-DC [VDC] to 300 VDC, via connection 29 to generate an output voltage via connection 30 to output device 14. For example, high voltage generator 24 may be an inverter-based voltage generator using a step-up transformer, or its equivalent. According to aspects of the invention, output device 14 is adapted to transmit the voltages generated by voltage generator 24 to one or more outlets 11. According to aspects of the invention, the voltage output by one or more outlets 11, and the variation of that voltage under the control and regulation of processor 12, is applied to one or more electrodes 9 mounted to a body having the target muscle, for example, human leg, arm, or back muscle, as described herein. Output device 14 may include a multiplexer 32, or a “demultiplexier” as known in the art, for example, whereby output device 14 may be adapted to selectively electrically couple any one or more of the output voltages 11 with the voltage introduced by connection 30 from high voltage generator 24.

In one aspect, output device 14 may be an inverter-based voltage generator, or its equivalent. In one aspect, output device 14 may also include a controller 34 adapted to monitor and/or regulate the voltages and/or current applied to electrodes 9 via outputs 11. For example, controller 34 may monitor and control the voltage output-by-output device 14 and/or monitor the current output by output device 14. In one aspect, controller 34 may be adapted to detect and/or regulate the current delivered to one or more electrodes 9, for example, adapted to detect and/or regulate the current on a pulse-by-pulse basis. For example, in on aspect, controller 34 may include a feedback loop where current directed to one or more electrodes 9 is detected, and then the voltage applied to the one or more electrodes 9 is regulated to achieve a desired current. For instance, when a target current is desired, for example, 2 mA, controller 34 may control the voltage output via outputs 11 to maintain the target current. Accordingly, in one aspect, should the detected current directed to one or more electrodes 9 vary from the target current, for example, due to a change in the impedance of the skin (for example, a reduction in impedance due to perspiration or moisture), controller 34 may vary the voltage (for example, decrease the voltage) to obtain the desired target current.

In one aspect, output device 14 may include an oscillator adapted to switch DC voltage into a transformer, for example, where the output voltage is handled in a fashion similar to a switched-mode power supply with a diode, for instance, a freewheeling Schottky diode, or its equivalent.

According to aspects of the invention, the one or more output voltages 11 may range from 10 VDC to 300 VDC, but are typically between about 40 VDC and about 100 VDC, for example, about 60 VDC. Also, the number N of outputs 11 may be unlimited, but may topically range from 2 outputs to 64 outputs; for example, N may range from 8 to 24 outputs, such as, 16 outputs. The number of outputs N may vary depending, among other things, upon the number of target muscles stimulated, the size of the target muscle, and/or the number of bodies treated by aspects of the invention.

In one aspect of the invention, processor 12 and/or controller 34 of output device 14 may be adapted to control or regulate the voltage output by outputs 11 and the frequency of the voltage output by outputs 11. For example, in one aspect, the variation in voltage polarity and/or magnitude may take the form of voltage “pulses” delivered at a frequency, for example, 1 kHz or more. In one aspect, the processor 12 and/or controller 34 of output device 14 may be adapted regulate and control the characteristics of the pulses, for example, the pulse magnitude, the pulse polarity, the pulse frequency, the pulse width, the time between pulses, and the number of pulses before or after polarity change, among other pulse characteristics. In one aspect, processor 12 and/or controller 34 of output device 14 may be adapted provide pulse-by-pulse variation or control of the voltage provided by one or more outputs 11.

It is envisioned that any one or more the connections, both the power and the control signal connections, shown in FIG. 1 may comprise one or more wired and/or wireless connections, for example, employing any conventional wireless protocol, such as, Bluetooth® and the like.

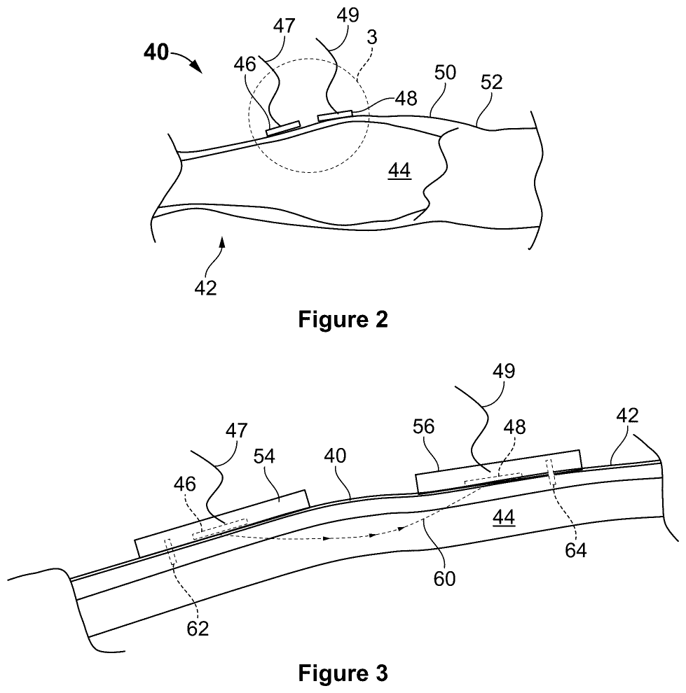

FIG. 2 is a schematic representation of an arrangement 40 of a cross-section of a portion of a body 42 having a target muscle 44 that can be stimulated according to aspects of the invention. As shown in FIG. 2, two or more electrodes 46 and 48 (for example, having substantially the same characteristics as electrodes 9 shown in FIG. 1) are mounted to the surface 50 of the skin 52 of the portion of the body 42. Though in the aspect shown in FIG. 2, the portion of the body 42 is depicted as the lower portion, or calf, of a human leg to facilitate illustration of the invention, it is envisioned that aspects of the invention may be used to stimulate any human or non-human muscle or muscle group. The muscles or muscle groups that may be stimulated include, but are not limited to, leg muscles, arm muscles, back muscles, torso muscles, hand muscles, foot muscles, buttocks muscles, neck muscles, and head muscles, among others.

As shown in FIG. 2, according to aspects of the invention, the two or more electrodes 46 and 48 are operatively connected to a source of voltage, for example, system 10 shown in FIG. 1, via wires or cables 47 and 49, respectfully. Typically, when a system such as system 10 shown in FIG. 1 is used, wires 47 and 49 may be electrically coupled or wired (that is, hardwired or wirelessly connected) to output device 14 shown in FIG. 1. When energized, electrodes 46 and 48 are adapted to provide a voltage across at least a portion of target muscle 44. FIG. 3 is a detailed cross section of the portion of the body 42 having electrodes 46 and 48 shown in FIG. 2 as identified by Detail 3 in FIG. 2.

As shown most clearly in FIG. 3, electrodes 46 and 48 may be mounted or encased in a packaging 54 and 56, respectively, for example, packages 54 and 56 may comprises flexible packaging or “patches” as known in the art. Packaging 54 and 56 may be mountable to the surface 50 of skin 52 adjacent to muscle 44. Packaging 54 and 58 may comprise a flexible material and be mounted to the surface 50 of skin 52 by conventional means, for example, removably mounted by means of an adhesive, as known in the art. In one aspect, electrodes 46 and 48 may comprise any form of conductive material, for example, a metal or a conductive plastic. Typically, electrodes 46 and 48 may comprise a conductive metal, for example, silver, a gold, or a copper. In one aspect, any conventional electrode material and electrode packaging construction may be used to contact the surface 50 and apply the desired voltages, as disclosed herein.

According to aspects of the invention, system 10 shown in FIG. 1 and electrodes 46 and 48 are adapted to expose target muscle 44 in FIG. 3 to generate a current flow-schematically represented by dashed line 60 shown in FIG. 3—in order to stimulate target muscle 44. Though the mechanism of current flow 60 is not well understood, it is envisioned that the naturally occurring ions or electrolytes in the tissue of skin 52 and target muscle 44 carry the electric current from one electrode, for example, electrode 46, to another electrode, for example, electrode 48, to stimulate the contraction of target muscle 44. According to aspects of the invention, any form of conductive fluid, for example, an electrolyte gel, may be applied between electrodes 46 and 48 and the surface 50 of skin 52 to enhance the exposure of target muscle 44 to the applied voltage.

As shown and described with respect to FIGS. 2 and 3, in one aspect, electrodes 46 and 48, and any other electrode disclosed herein, may sufficiently contact the surface 50 of skin 52 to expose target muscle 44 to the applied voltage and transmit the current 60. That is, in one aspect, electrodes 46 and 48 may operate “transcutaneously” with respect to skin 52. However, in other aspects of the invention, electrodes 46 and 48, and any other electrode disclosed herein, may penetrate the surface 50 of skin 52, for example, at least partially penetrate skin 52, to expose target muscle 44 to the applied voltage and transmit the current 60. That is, in one aspect, electrodes 46 and 48 may operate “subcutaneously” with respect to skin 52. For example, in one aspect electrodes 46 and 48 may comprise one or more projections or “needles” (for example, micro needles) 62 and 64 that penetrate skin 42 to expose target muscle 44 to the desired voltages disclosed herein. In one aspect, one or more projections or needles 62 and 64 may penetrate into target muscle 44 and expose target muscle 44 to the desired voltages disclosed herein.

In FIGS. 2 and 3, aspects of the invention are shown with electrodes 46 and 48 located in packaging or “patches” 54 and 56 in order to facilitate the illustration of one aspect of the invention. However, it is envisioned that two or more electrodes 46 and 48 may be provided in a broad range of housings, packaging, or “wearables” and the like while providing the benefits of the present invention. For example, two or more electrodes 46 and 48 may be provided in a “patch” having a plurality of electrodes 46 and 48, for example, an array of electrodes 46 and 48. In another aspect, two or more electrodes 46 and 48 may be provided in a wearable housing, for example, a housing retained by a band or strap adapted to be mounted about the target muscles. For instance, in one aspect, two or more electrodes 46 and 48 may be mounted in a wristwatch type device. In another aspect, two or more electrodes 46 and 48 may be provided in a wearable garment, for example, in a shirt, in a pair of pants, or in a band adapted to be wrapped about body part of the target muscle.

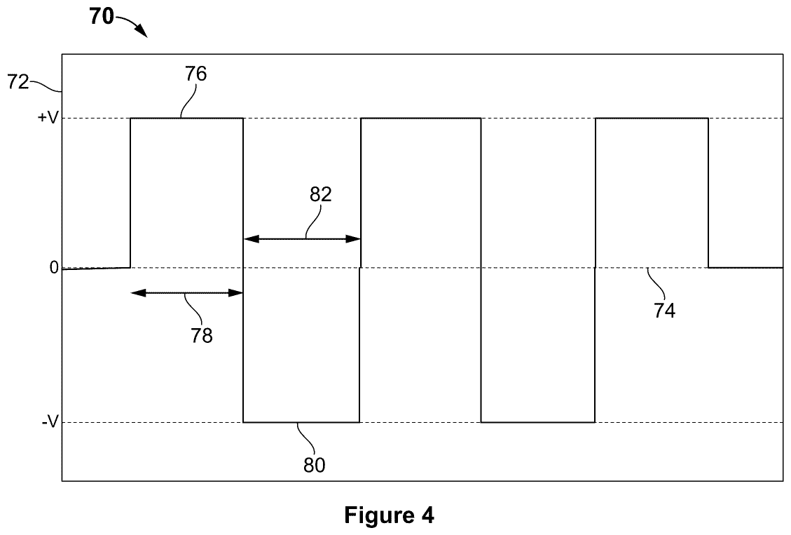

According to aspects of the invention, system 10 of FIG. 1 is used to provide a voltage, for example, a DC voltage, of varying polarity to stimulate a target muscle, for example, target muscle 44 in FIG. 3. FIGS. 4 through 7 provide representative illustrations of the voltages, the variation in voltages, and the variation in voltage polarity that can be provided by aspects of the invention, for example, by system 10 shown in FIG. 1 and electrodes 46 and 48 shown in FIGS. 2 and 3, according to aspects of the invention.

FIG. 4 is a representative plot 70 of voltage as a function of time that may be applied to electrodes to stimulate a target muscle according to an aspect of the invention. The plot 70 illustrates that, in one aspect, the voltage applied may be repeatedly varied in polarity and/or magnitude, for example, substantially immediately varied, for fixed durations of time or varying durations of time.

In FIG. 4, the ordinate 72 of plot 70 represents voltage, specifically, DC voltage, and the abscissa 74 represents time, for example, millisecond or minutes. As shown in FIG. 4, in one aspect, the voltage that may be applied across a target muscle by aspects of the invention may comprise a first voltage 76, for example, 100 VDC, having a first polarity (for example, positive (+)) for a first time period 78, for example, 5 milliseconds, then followed (for example, substantially immediately followed) by a second voltage 80, for example, 100 VDC, having a second polarity (for example, negative (−)), opposite the first polarity, for a second time period 82, for example, 5 milliseconds, to expose the target muscle to sufficient stimulation.

According to aspects of the invention, it is understood at this time that the variation in the polarity of the voltages shown in FIG. 4 may minimize or prevent the undesirable local polarization and ion migration that characterizes the prior art. For example, it is believed the change in polarity of the voltages shown in FIG. 4, and throughout this disclosure, counter acts any charge accumulation that may occur compared to the application of a continuous voltage or a voltage that does not change polarity. Accordingly, it is believed that aspects of the invention minimize or prevent the undesirable ion migration that characterizes some prior art methods.

In one aspect, the voltage 76 applied over the time period 78 and the voltage 80 applied over the time period 82, and any voltages applied over a time period disclosed herein, may be referred to as voltage “pulses.” For example, plot 70 in FIG. 4 may be described as illustrating a plurality of positive polarity pulses, and each of the positive polarity pulses are followed substantially immediately by a negative polarity pulse. As will be described herein, the sequence and timing of positive pulses and negative pulses may be varied from what is shown in FIG. 4 while still providing the benefits of the present invention.

In one aspect, the voltages that produce sufficient stimulation comprise a voltage at which the target muscle at least partially contracts. In another aspect, the voltages that produce sufficient stimulation comprise a voltage at which the target muscle substantially completely contracts.

According to aspects of the invention, first voltage 76 and second voltage 80 may range from about 10 VDC to about 300 VDC. Also, first time period 78 and second time period 82 may range from about 1 microsecond [μs] to about 10 milliseconds [ms], but are typically between about 5 μs and about 1 ms. For example, in one aspect, the frequency of the variation in voltage shown in FIG. 4 may range from about 200 hertz [Hz], or 0.2 kilohertz [KHz], to about 20 kHz, but is typically between about 1 kHz and 10 KHz. Accordingly, first time period 78 and second time period 82 may range from about 0.005 seconds (that is, 5 ms) to about 0.00005 seconds (that is, 50 μs).

Also, it is believed that this relatively high frequency, for example, greater than 1 kHz of the aspect shown in FIG. 4, and of any aspect disclosed herein, can be effective in reducing the capacitive impedance, XC, of the skin being treated, and thus reduce the likelihood for pain and increase the effectiveness of the stimulation of the target muscle.

As shown in FIG. 4, the sequence and timing of first voltage 76 and second voltage 80 may be repeated, for example, repeated a plurality of times. For example, in one aspect, for the sequence and timing shown in FIG. 4 (or for any sequence and timing disclosed herein), the pulses may be applied for 1-5 seconds, then the voltage pluses may stop (for instance, for a “rest period”), for example, for 5 seconds to 5 minutes, and then the pulses applied again. This application-rest-application sequence may be repeated multiple times as desired or required. In one aspect, the duration of the treatment represented by plot 70 in FIG. 4 (or for any sequence and timing disclosed herein) may be repeated for a total treatment time from 2 seconds to 30 minutes, but is typically the duration of treatment is from 5 minutes to 15 minutes.

Though not shown in FIG. 4, it is envisioned that first voltage 76 and second voltage 80 may be substantially constant or may vary in magnitude between pulses. For example, in one aspect, first voltage 76 and/or second voltage 80 may vary linearly (increasing or decreasing), quadratically (increasing or decreasing), by any time-dependent variation, or erratically. In addition, it is also envisioned that time periods 78 and 82 may be substantially constant or may also vary in duration.

In the aspect of the invention shown in FIG. 4 (and in other FIGs. herein), for the sake of ease of illustration, the variation in voltage magnitude and polarity is shown as substantially instantaneous, for example, in a fashion representing step functions. However, it is envisioned that the change in magnitude or polarity may not comprise such instantaneous changes, but due to the system response or as desired, the voltage and polarity may not vary instantaneously, but gradually, for example, linearly. For example, in one aspect, the variation from first voltage 76 and second voltage 80 (or any voltage variation disclosed herein) may vary linearly, quadratically, or at any defined or undefined time dependent rate while providing the advantages and benefits of aspects of the present invention.

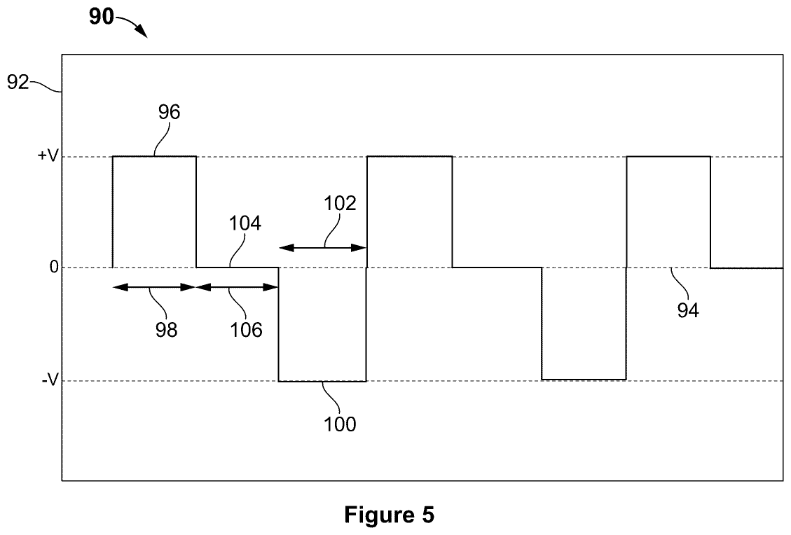

FIG. 5 is a representative plot 90 of voltage as a function of time that may be applied to electrodes to stimulate a target muscle according to another aspect of the invention. The plot 90 illustrates that, in one aspect, the voltage applied may be repeatedly varied in polarity and/or magnitude, for example, with time intervals between the variations. The time intervals may be fixed durations of time or varying durations of time.

In FIG. 5, the ordinate 92 of plot 90 represents voltage, specifically, DC voltage, and the abscissa 94 represents time, for example, millisecond or minutes. As shown in FIG. 5, in one aspect, the voltage that may be applied across a target muscle by aspects of the invention may comprise a first voltage 96 having a first polarity (for example, positive (+)) for a first time period 98 followed by a second voltage 100, for example, 100 VDC, having a second polarity (for example, negative (−)), opposite the first polarity, for a second time period 102 to expose the target muscle to sufficient stimulation. In the aspect, as shown in FIG. 5, after the first voltage 96 and before the second voltage 100, the voltage applied to the electrodes may comprise a third voltage 104, for example, a voltage of substantially zero voltage, for a third time period 106. For example, in one aspect, the third voltage 104 may be provided by de-energizing the at least two electrodes to expose the target muscle to little or no voltage. In one aspect, the third voltage may be non-zero, and may be of positive or negative polarity. In one aspect, the voltages that produce sufficient stimulation comprise a voltage at which the target muscle at least partially contracts. In another aspect, the voltages that produce sufficient stimulation comprise a voltage at which the target muscle substantially completely contracts.

According to aspects of the invention, first voltage 96, the second voltage 100, and the third voltage 104 in FIG. 5 may range in voltage as first voltage voltages 76 and second voltage 80 may range, for example, from about 10 VDC to about 300 VDC. Also, first time period 98, the second time period 102, and the third time period 106, like time periods 78 and 82, may range from about 1 μs to about 10 ms. In one aspect, the frequency of the variation in voltage shown in FIG. 5 may range from about 500 Hz to about 20 kHz, but is typically between about 1 kHz and 10 KHz.

As shown in FIG. 5, the sequence and timing of first voltage 96, second voltage 100, and third voltage 102 may be repeated, for example, repeated a plurality of times. In one aspect, the duration of the treatment represented by plot 90 in FIG. 5 may be repeated for a total treatment time from 2 seconds to 30 minutes, but is typically the duration of treatment is from 1 minute to five minutes.

Though not shown in FIG. 5, it is envisioned that first voltage 96 and second voltage 100 may be substantially constant or may vary in magnitude between pulses. For example, in one aspect, first voltage 96 and/or second voltage 100 may vary linearly (increasing or decreasing), quadratically (increasing or decreasing), by any time-dependent variation, or erratically. In addition, it is also envisioned that time periods 98, 102, and 106 may be substantially constant or may also vary in duration.

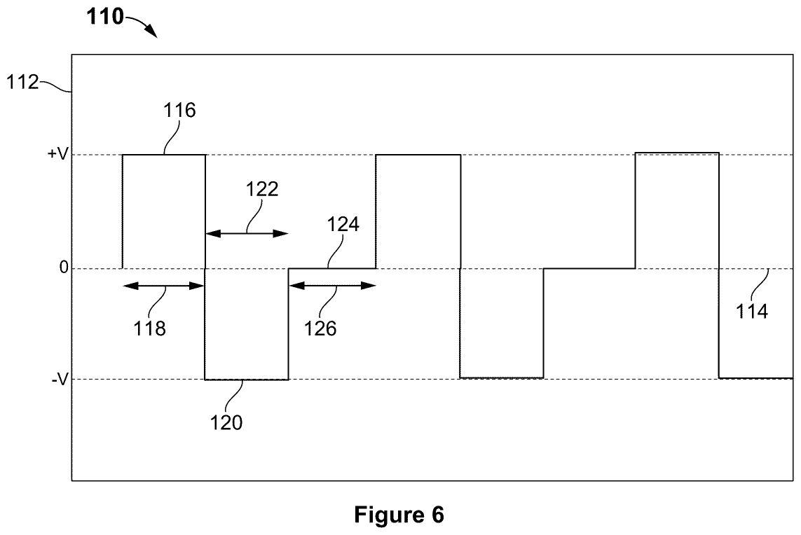

FIG. 6 is a representative plot 110 of voltage as a function of time that may be applied to electrodes to stimulate a target muscle according to another aspect of the invention. The plot 110 illustrates that, in one aspect, the voltage applied may be repeatedly varied in polarity and/or magnitude, for example, with time intervals between the variations, for fixed durations of time or varying durations of time.

In FIG. 6, the ordinate 112 of plot 110 represents voltage, specifically, DC voltage, and the abscissa 114 represents time, for example, millisecond or minutes. As shown in FIG. 6, in one aspect, the voltage that may be applied across a target muscle by aspects of the invention may comprise a first voltage 116 having a first polarity (for example, positive (+)) for a first time period 118 followed by a second voltage 120 having a second polarity (for example, negative (−)), opposite the first polarity, for a second time period 122 to expose the target muscle to sufficient stimulation. In the aspect shown in FIG. 6, after the first voltage 116 and the second voltage 120, the voltage applied to the electrodes may comprise a third voltage 124, for example, a voltage of substantially zero voltage, for a third time period 126. For example, in one aspect, the third voltage 124 may be provided by de-energizing the at least two electrodes to expose the target muscle to little or no voltage. In one aspect, the third voltage may be non-zero, and may be of positive or negative polarity. In one aspect, the voltages that produce sufficient stimulation comprise a voltage at which the target muscle at least partially contracts. In another aspect, the voltages that produce sufficient stimulation comprise a voltage at which the target muscle substantially completely contracts.

According to aspects of the invention, first voltage 116, the second voltage 120, and the third voltage 124 in FIG. 6 may range in voltage as first voltage voltages 76 and second voltage 80 may range, for example, from about 10 VDC to about 300 VDC. Also, first time period 118, second time period 122, and the third time period 126, like time periods 78 and 82, may range from about 1 μs to about 10 ms. In one aspect, the frequency of the variation in voltage shown in FIG. 6 may range from about 500 Hz to about 20 kHz, but is typically between about 1 kHz and 10 KHz.

As shown in FIG. 6, the sequence and timing of first voltage 116, second voltage 120, and third voltage 124 may be repeated, for example, repeated a plurality of times. In one aspect, the duration of the treatment represented by plot 110 in FIG. 6 may be repeated for a total treatment time from 2 seconds to 30 minutes, but is typically the duration of treatment is from 1 minute to five minutes.

Though not shown in FIG. 6, it is envisioned that first voltage 116 and second voltage 120 may be substantially constant or may vary in magnitude between pulses. For example, in one aspect, first voltage 116 and/or second voltage 120 may vary linearly (increasing or decreasing), quadratically (increasing or decreasing), by any time-dependent variation, or erratically. In addition, it is also envisioned that time periods 118, 122, and 126 may be substantially constant or may also vary in duration.

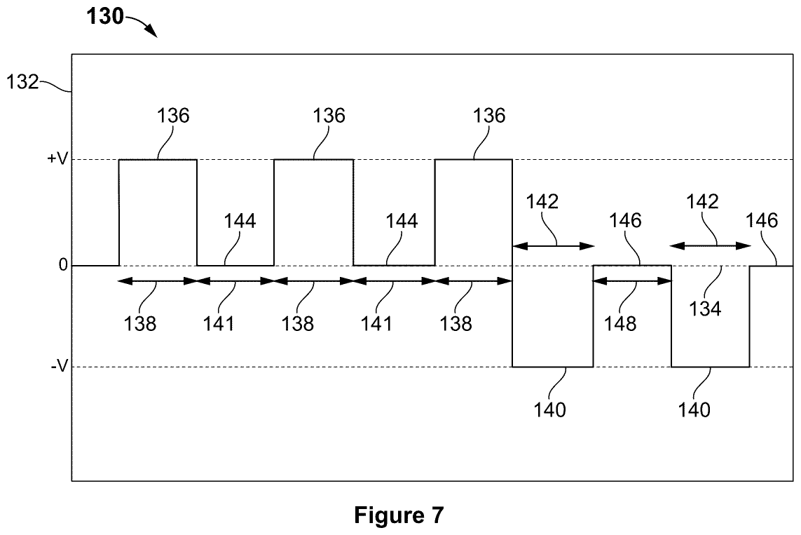

FIG. 7 is a representative plot 130 of voltage as a function of time that may be applied to electrodes to stimulate a target muscle according to another aspect of the invention. The plot 130 illustrates that, in one aspect, the voltage applied may be repeatedly applied as “pulses” of a first voltage at a first polarity and then repeatedly applied as “pulses” of a second voltage at a second polarity, opposite the first polarity, for example, with time intervals between the pulses. The time intervals between pulses may be of fixed durations of time or varying durations of time.

In FIG. 7, the ordinate 132 of plot 130 represents voltage, specifically, DC voltage, and the abscissa 134 represents time, for example, millisecond or minutes. As shown in FIG. 7, in one aspect, the voltage that may be applied across a target muscle may comprise repeatedly applying a first voltage 136 having a first polarity (for example, positive (+)) for a first time period 138 followed by repeatedly applying a second voltage 140 having a second polarity (for example, negative (−)), opposite the first polarity, for a second time period 142 to expose the target muscle to sufficient stimulation. In the aspect shown in FIG. 7, between each first voltage 136, a third voltage 144, for example, substantially zero voltage (though in some aspects, the third voltage 144 may be non-zero of either polarity), may be applied for a third time period 141. Similarly, as shown in FIG. 7, between each second voltage 140, a fourth voltage 146, for example, substantially zero voltage (though in some aspects, the third voltage 146 may be non-zero of either polarity) may be applied for a fourth time period 148. For example, in one aspect, the third voltage 144 and the fourth voltage 146 may be provided by de-energizing the at least two electrodes to expose the target muscle to little or no voltage. In one aspect, the voltages that produce sufficient stimulation comprise a voltage at which the target muscle at least partially contracts. In another aspect, the voltages that produce sufficient stimulation comprise a voltage at which the target muscle substantially completely contracts.

Though not shown in FIG. 7, it is envisioned that first voltage 136 and second voltage 140 may be substantially constant or may vary in magnitude between pulses. For example, in one aspect, first voltage 136 and/or second voltage 140 may vary linearly (increasing or decreasing), quadratically (increasing or decreasing), by any time-dependent variation, or erratically. In addition, it is also envisioned that time periods 138, 141, 142, and 148 may be substantially constant or may also vary in duration.

According to aspects of the invention, first voltage 136, the second voltage 140, the third voltage 144, and the fourth voltage 146 in FIG. 7 may range in voltage as first voltage 76 and second voltage 80 may range, for example, from about 10 VDC to about 300 VDC. Also, time period 138, 141, 142, and 148, like time periods 78 and 82 disclosed herein, may range from about 1 μs to about 10 ms. In one aspect, the frequency of the variation in voltage shown in FIG. 7 may range from about 500 Hz to about 20 kHz, but is typically between about 1 kHz and 10 KHz.

In one aspect, the sequence and timing voltages shown in FIG. 7 (for example, the pulses) may be repeated, for example, repeated a plurality of times. Though first voltage 136 in FIG. 7 is shown repeated three times, it is envisioned that first voltage 136 may be repeated 10 or more times or hundreds or thousands of times, for example, at a frequency ranging, for example, from 1 kHz to 10 KHz. Similarly, it is envisioned that second voltage 140 may be repeated 10 or more times or hundreds or thousands of times, for example, at a frequency ranging, for example, from 1 kHz to 10 KHz. In one aspect, the duration of the treatment represented by plot 130 in FIG. 7 may be repeated for a total treatment time from 1 second to 30 minutes, but is typically the duration of treatment is from 1 minute to five minutes.

The voltage variations and timing shown in FIGS. 4 through 7 are presented as typical voltage applications that may be used for target muscle stimulation according to aspects of the invention. However, it is envisioned that those of skill in the art will conceive of myriads of other variations and timing sequences not illustrated herein, but for the sake of brevity are not presented herein.

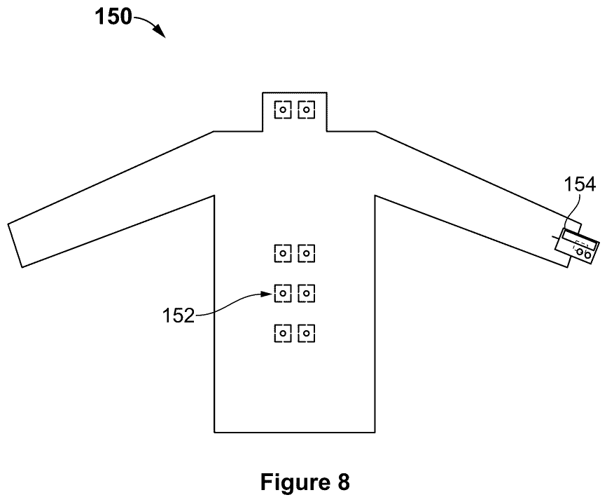

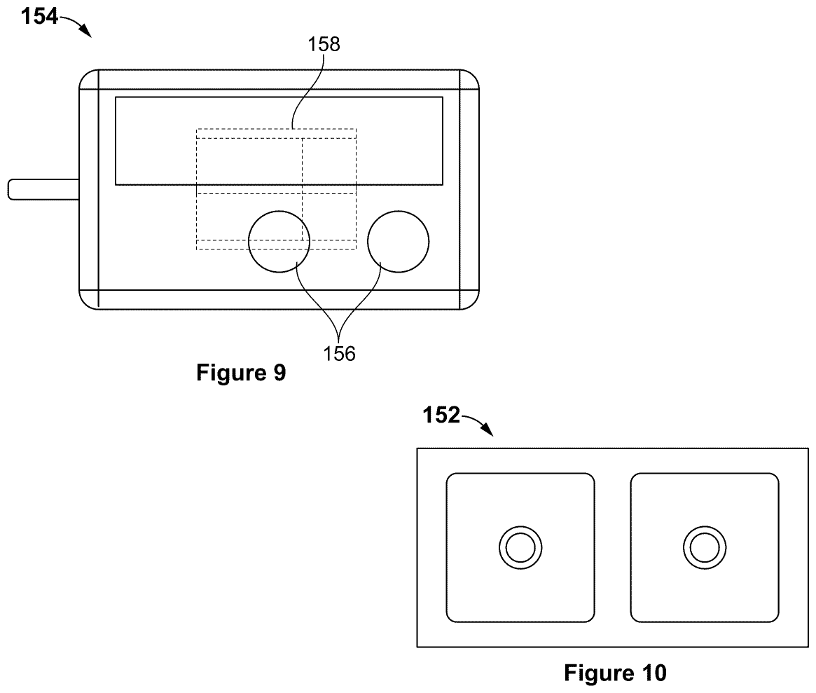

FIG. 8 is a representative illustration of the wearable device 150 with electrodes 152, controller 154 with power On/Off buttons 156, and alkaline batteries 158 (shown in FIGS. 9 and 10). The wearable device 150 may be a shirt. Herein, it is a view of electrodes 152 on the inner side (fabric liner) of the shirt. The electrode connections such as wire (not shown in figure) to the controller 154 may be embedded in the shirt fabric. FIG. 9 is a representative illustration of the controller 154 with On/Off buttons 156 and alkaline batteries 158. FIG. 10 is a representative illustration of the electrodes 152.

As shown in FIGS. 8 through 10, the wearable device 150 is incorporated with hardware components including: electrodes 152, micro-electronics (not shown in figure), controller 154 with on/off buttons 156, and alkaline batteries 158, developed with software including algorithms and firmware. According to aspects of the invention, the controller 154 may be positioned at the wrist/hand of the user. A real-time firmware is also embedded on the controller 154. The wearable device 150 recited with the system 10 (shown in FIG. 1), embodies an innovative method utilizing functional electrical stimulation (FES).

In one aspect, wearable device 150 incorporated with electrodes 152 for pain relief with wire (not shown in figure) along arm to the controller 154. For example, here the solution is, wearing it as shirt liner (base layer) and wire (not shown in figure) routed along the sleeves to the wrist/hand of the user. Further, this wearable device 150 is specifically designed for: (1) neck/back pain during operational conditions, and flight environment on military equipment, especially naval environments. One key aspect of this innovation is: the wearable device 150 is powered by alkaline batteries 158 (non-lithium), which is to be used in aircraft/flight environment. Lithium batteries are dangerous in-flight environment, which can produce dangerous heat levels, cause ignition, short circuit very easily, and cause inextinguishable fires. This inventive aspect provides solution for neck/back pain of the user during military or normal passenger flight operations. In addition, the wearable device 150 could also be used for all environmental conditions in civilian markets.

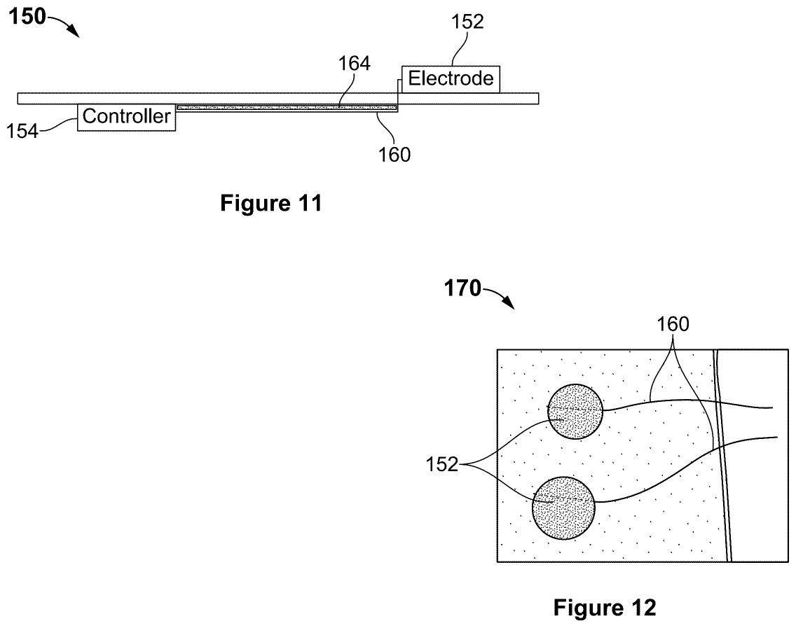

FIG. 11 exemplarily illustrates an arrangement of components of a wearable device 150, according to an embodiment of the present invention. In one embodiment, the wearable device 150 is an apparel. Generally, the apparel includes an exterior side and an interior side. The interior side refers to a side that contacts the body of the wearer. The exterior side refers to a side that is exposed to the environment. The device 150 comprises one or more electrodes 152 and a controller 154 connected to the electrode 152. The electrodes 152 are disposed at the interior side of the apparel and the controller 154 is disposed at the exterior side of the apparel.

The device 150 further comprises at least one first conductive thread 160. The first conductive thread 160 extends between the controller 154 and the electrodes 152 to electrically connect the controller 154 and the electrodes 152. The first conductive thread 160 extends at the exterior side of the apparel and electrically connects the controller 154 to the electrode 152. The device 150 is constructed to prevent the first conductive thread 160 from contacting the body. Further, the device 150 is constructed in such a way the first conductive thread 160 only contacts the electrodes 152. In one embodiment, the first conductive thread 160 has low resistance. The first conductive thread 160 is used to carry signal from the controller 154 to the electrodes 152. In one embodiment, controller 154 uses low-voltage, for example, 6V. Further, the present invention uses Functional Electrical Stimulation (FES) devices. With FES devices, pulse width is commonly between 150 μs to 30 μs. Further, fuses are used to prevent a high-current circuit situation. A high-current occurs when something has been shorted. When a high-current situation occurs, the fuse will blow thus protecting the hardware and person. The controller 154 is designed with current limiting circuitry. In one embodiment, transient voltage suppressors are used to protect circuits from electrostatic discharges.

In one embodiment, the electrodes 152 are constructed using an embroidery machine with a second conductive threads. In one embodiment, the second conductive threads include high resistance. However, the resistance is reduced by increasing the area of the material. This achieved with the circular shape of the electrodes 152. In one embodiment, the electrodes 152 of circular shape are provided at the apparel. In another embodiment, the electrodes 152 could have any other shapes.

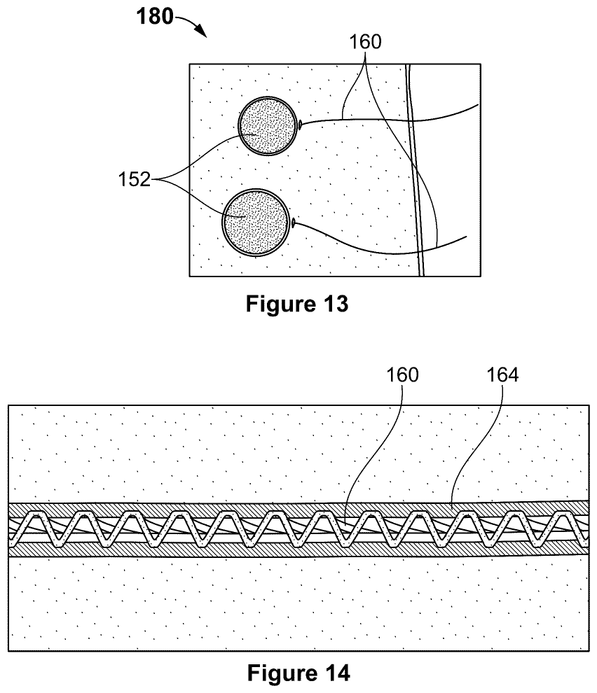

Referring to FIG. 12, a segment 170 of interior side of the apparel is sewn with at least two electrodes 152, according to an embodiment of the present invention. In another embodiment, one or more electrodes 152 could be disposed at the apparel. FIG. 13 exemplarily illustrates a segment 180 of exterior side of the apparel, according to an embodiment of the present invention. Further, first conductive threads 160 extend at the exterior side of the apparel.

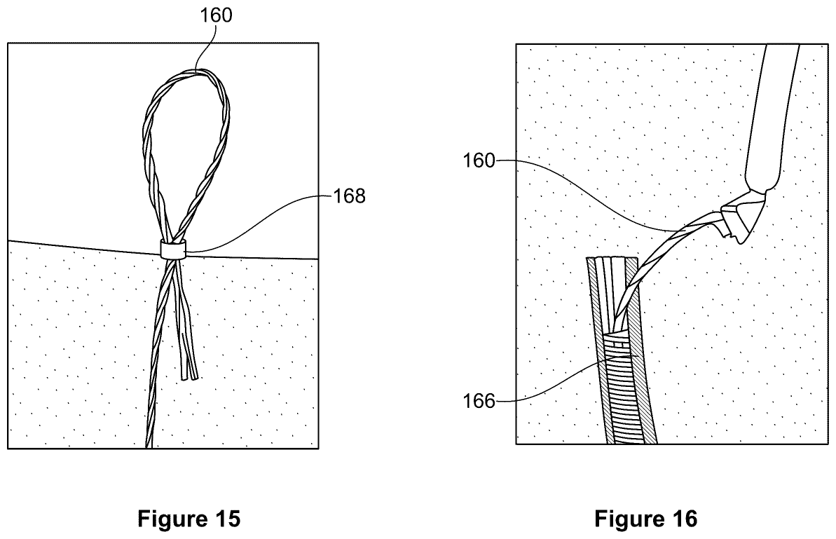

Referring to FIG. 14, the device 150 further comprises an insulation assembly 164. The insulation assembly 164 comprises electrical insulation material, which is woven over the first conductive thread 160. Referring to FIG. 15, the first conductive thread 160 is pulled through bead 168 to form a loop.

Referring to FIG. 16, the first conductive thread 160 passing beyond the insulation assembly 164, according to an embodiment of the present invention. The insulation assembly 164 limits the current flow to the first conductive thread 160. The insulation assembly 164 and the first conductive thread 160 enclosed by the cover 166. FIG. 14 to FIG. 16 is detailly explained in foregoing paragraphs.

The wearable device 150 is an electrically conductive bio-shirt that can be worn like a normal undergarment. The bio-shirt is adapted to conform the body of the wearer. In one embodiment, the conductive threads are electrically conductive, flexible enough to conform to the body, has a characteristic of the normal fabric, machine washable and could be used with a sewing machine or an embroidery machine.

The material properties of the of the second conductive thread, shape and diameter of the electrodes 152 and density (thread count) of the electrodes 152 are chosen to significantly reduce the resistivity of the electrode 152; thus, allowing sufficient current flow to stimulate the muscle. The relationship between resistance and the second conductive thread cross-sectional area can be expressed as follows:

$$R = ( ρ L ) / A$$

Where, ρ is the second conductive thread resistivity, L is the length and A is the cross-sectional area. In general, the resistance is always inversely proportional to the cross-sectional area of the conductor which can be expressed as follows,

$$R ∝ ( 1 ) / A$$

This ratio indicates the second conductive thread resistivity, and the cross-sectional surface area are the two major factors in the determining the electrode's resistance. The resistance issue was solved by using conductive thread. These threads have resistance <300 Ohm/m.

When using conductive thread with an embroidery machine to create the electrode 152 with the following parameters, including diameter 38 mm and embroidery thread count 5000. The surface area of the thread has been increased and therefore the resistance of the electrode 152 has been reduced to approximately 0.7 Ohms.

Further, the conductive traces or first conductive threads 160 are used to carry electric current from the controller 154 to the electrodes 152. Contrary to the thin and flexible second conductive thread used to construct the electrodes 152, the conductive thread traces are strong and rigid enough to handle the movement of the body. Also, the first conductive thread 160 have low resistivity to prevent dissipation of the electrical current from the controller 154 as heat. This conductive material has low resistivity, 10 Ohms per foot. To further reduce the Ohms/foot, two strands of first conductive thread 160 run in parallel to reduce the Ohms by half.

Considering the Electrodes and the Conductive traces are to different materials, a method was developed to ensure the two materials will remain attached to each other during normal use. Also, the interface between the two materials must have low impendence connection. In other words, the electrical current must be able to freely flow from one material to the other material. This was achieved during construction by overlaying the conductive trace thread or first conductive thread 160 with the electrode thread or the second conductive thread during the embroidery process.

Further, the first conductive threads 160 are insulated, which is explained as follows. first conductive threads 160 are transferred to the outside of the shirt. The outside portion of the shirt does not contact with the body. Note that conductive first conductive threads 160 is attached to second conductive thread on the inside half of the shirt as shown in FIG. 12. Then the loose end of first conductive thread 160 is threaded to the outside half of the shirt as shown in FIG. 13. With this configuration, the shirt serves as level one insulation. The first conductive threads 160 is held in the cradle with a zig-zag pattern created with a sewing machine, which is shown in FIG. 14. The final step of insulation is to use an embroidery thread to cover 166 the first conductive threads 160 and the insulation assembly 164, as shown in FIG. 16. This prevents first conductive threads 160 from making fan electrical contact with anything that may be touching the outside of the shirt.

Another challenge of the present invention is disclosed as follows. Solder is a low-melting alloy used for joining less fusible metals. Typically, in electronics solder is used to wires and discrete components to a PCB (Printed Circuit Board). The solder process makes it possible for electrical current to flow from one device to another. Without the ability to solder-connect the conductive threads to the controllers' PCB, we had to develop a reliably method to transition from conductive thread to standard wiring at the PCB to interact with the controller. This challenge is addressed and explained as follows.

Due to the properties of first conductive thread 160, it cannot be soldered. Therefore, a technique was developed to transition from a conductive thread to a standard wire at the controller interface. The technique involves pulling the stainless-steel first conductive thread 160 through bead 168 to form a loop, at step 1, which is shown in FIG. 15. At step 2, the controller wire is inserted through the loop of first conductive thread 160 (also referred as conductive thread loop). At step 3, the conductive thread loop is pulled close. At step 4, the bead 168 is crimped; thus, securing the first conductive thread 160 and the controller wire. At step 5, the interface that encompass the bead, controller wire and the conductive thread is soldered. The result is shown in FIG. 16.

Further, For the electrical current to reliably flow from the controller 154 to the electrodes 152 and finally to the skin, a good physical contact between the electrodes 152 and the skin must be achieved. This is being addressed by constructing the shirt to be formed to “hug” the board. This will be like a compression garment. Also, elastic bands are placed near the electrode 152 site to encourage the electrode 152 to make good physical contact with the body.

Another challenge faced by the present is electrical Contact between the electrodes 152 and the skin. Basically, poor electrical contact between the electrodes and the skin will resemble a high impedance interface which will result in poor flow of electrical current. Typically, poor electrical connection occurs when the skin is dry. The present invention addresses this issue with gel substances and/or conductive film.

In contrast to existing “Transcutaneous Electrical Nerve Stimulation” (TENS) technology using direct current (D/C) and existing “Functional Electrical Stimulation” (FES) technology, an electrical muscle stimulation technology incorporated in the wearable device 150 uses alternating current (A/C). The A/C stimulation, for example, at a higher frequency and lower power, provides effective muscle stimulation (and function) that is painless (compared to higher power D/C-type stimulation) and can be applied to stimulate muscle function in a broad range of applications.

According to aspects of invention, A/C stimulation is more effectively transmitted “transcutaneously” to a target muscle. Moreover, since the A/C stimulation can more effectively access the subcutaneous muscle tissue, actual “functional stimulation” (muscle contraction) can be initiated. In contrast to conventional D/C functional stimulation, A/C functional stimulation can be provided without the increased power and the pain that the increased power causes, which characterizes D/C stimulation. In effect, the present invention also proposes methods and devices for painless, lower-power initiation and control of muscle contraction.

The limits of TENS and traditional FES for patients who have skin sensation are limited by their ability to withstand the pain of the stimulation and, assuming that pain is directly proportional to the voltage being applied to the skin, then the pain will be reduced by this proposed invention because a lower voltage will be needed to create a muscle-stimulating FES pulse of a given current, thereby the effective impedance of the skin is reduced by using an AC waveform in the delivery of the stimulation. Henceforth, the present invention achieves the same FES current using a lower voltage.

According to aspects of the invention, a system of electrode outputs is disclosed. The microcontroller could make the electrode pair any two (or more) electrodes so the present invention provides solution that can generate more complicated stimulations. The present invention could lessen the pain aspects of FES by spreading the delivered current over multiple electrodes.

Though according to some aspects of the invention disclosed herein, a patient may be treated with voltage and voltage variation alone, in other aspects, the voltage variation stimulation disclosed herein may be supplemented with exposure to heat, humidity, and/or message. One or more of these supplemental treatments may enhance the effectiveness of the voltage stimulation disclosed herein. The supplemental treatment may be applied with the voltage stimulation disclosed herein, for example, at substantially the same time, or intermittently with voltage stimulation.

According to aspects of the invention, the variation in voltage and polarity stimulation disclosed herein may be effective in stimulating muscles that otherwise do not respond to a patient's nervous system. For example, aspects of the invention may be effective in stimulating muscles affected by spinal cord injury, stroke, and/or muscle sclerosis, among other ailments. However, aspects of the invention overcome the disadvantages of the prior art, including undesirable ion migration and the potential for pain.

While various embodiments have been described above, it should be understood that they have been presented by way of example and not limitation. It will be apparent to persons skilled in the relevant art that various changes in form and detail can be made therein without departing from the spirit and scope. After reading the above description, it will be apparent to one skilled in the relevant art how to implement alternative embodiments.

In addition, it should be understood that any FIGs., which highlight the functionality and advantages, are presented for example purposes only. The disclosed methodology and system are each sufficiently flexible and configurable such that they may be utilized in ways other than that shown. Although the term “at least one” may often be used in the specification, claims and drawings, the terms “a”, “an”, “the”, “said”, etc. also signify “at least one” or “the at least one” in the specification, claims and drawings. While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

What is claimed is:

1. A method for stimulating a muscle, the method comprising:
a) contacting a surface of the skin in proximity to a target muscle with at least two electrodes;
b) energizing the at least two electrodes to expose the target muscle to a first voltage having a first polarity for a first time period; and
c) energizing the at least two electrodes to expose the target muscle to a second voltage having a second polarity, opposite the first polarity, for a second time period;
wherein varying the polarity of the voltage to which the target muscle is exposed is sufficient to stimulate the target muscle.

2. The method of claim 1, wherein the first voltage ranges from 10 VDC to 300 VDC.

3. The method of claim 1, wherein the first voltage comprises a frequency of 1 kHz to 10 KHz.

4. The method of claim 1, wherein the method further comprises, between b) and c), de-energizing the at least two electrodes to expose the target muscle to little or no voltage.

5. The method of claim 1, wherein the method further comprises, after c), de-energizing the at least two electrodes to expose the target muscle to little or no electrical energy.

6. The method of claim 1, wherein b) is repeated a plurality of times prior to c).

7. The method of claim 1, wherein c) is repeated a plurality of times.

8. The method of claim 1, wherein contacting the surface of the skin in proximity to a target muscle comprises mounting the at least two electrodes in one or more packages, and mounting the one or more packages to the surface of the skin.

9. The method of claim 1, wherein the method further comprises monitoring a current directed to the at least two electrodes and varying at least one of the first voltage and the second voltage in response to the current.

10. The method of claim 1, wherein varying at least one of the first voltage and the second voltage in response to the current comprises varying at least one of the first voltage and the second voltage to maintain a target current.

11. A system for stimulating a muscle, the system comprising:
at least two electrodes adapted to contact a surface of the skin in proximity to a target muscle; and
an output device having at least two outputs operatively connected to the at least two electrodes, wherein the output device is configured to provide at one of the at least two outlets a first voltage having a first polarity for a first time period, and to provide at another of the at least two outlets a second voltage having a second polarity, opposite the first polarity, for a second time period;
wherein varying the polarity of the voltage to which the target muscle is exposed is sufficient to stimulate the target muscle.

12. The system of claim 11, wherein the system further comprises a high voltage generator operatively connected to the output device to provide the first voltage and the second voltage.

13. The system of claim 11, wherein the first voltage and the second voltage range from 10 VDC to 300 VDC.

14. The system of claim 11, wherein the output device is further adapted to vary a frequency of at least one of the first voltage and the second voltage.

15. The system of claim 14, wherein the frequency ranges from 1 kHz to 10 kHz.

16. A wearable device having the system recited in claim 11.

17. The wearable device of claim 16, wherein the wearable device comprises a patch having an adhesive adapted to at least temporarily adhere the patch to human skin, and wherein the wearable device is mounted in at least one of a garment, gear, and a textile.

18. The system of claim 11, wherein the system further comprises a controller adapted to monitor a current directed to the at least two electrodes and adapted to vary at least one of the first voltage and the second voltage in response to the current.

19. The system of claim 11, wherein the controller is adapted to vary at least one of the first voltage and the second voltage to maintain a target current.

20. A system for stimulating a muscle, the system comprising:
a wearable device comprising a textile portion and at least two electrodes, wherein the textile portion comprises an interior side adapted to contact a skin surface of a wearer and an exterior side, wherein the electrodes are disposed at the interior side of the textile portion and adapted to contact the skin surface in proximity to a target muscle;
an output device having at least two outputs operatively connected to the at least two electrodes, wherein the output device is configured to provide at one of the at least two outlets a first voltage having a first polarity for a first time period, and to provide at another of the at least two outlets a second voltage having a second polarity, opposite the first polarity, for a second time period;
a controller disposed at the exterior side of the textile portion, the controller is connected to the output device and the electrodes, wherein the controller is adapted to monitor a current directed to the at least two electrodes and adapted to vary at least one of the first voltage and the second voltage in response to the current;
at least one first conductive thread extends from the controller to the wearable device at the exterior surface of the textile portion to electrically connect the controller to the wearable device,
wherein varying the polarity of the voltage to which the target muscle is exposed is sufficient to stimulate the target muscle, and
a high voltage generator operatively connected to the output device to provide the first voltage and the second voltage, and an insulation assembly comprising electrical insulation material woven over the first conductive thread.

21. The system of claim 20, wherein the output device is further adapted to vary a frequency of at least one of the first voltage and the second voltage.

22. The system of claim 20, wherein the controller is adapted to vary at least one of the first voltage and the second voltage to maintain a target current.
